(19) United States
(12) Patent Application Publication
(10) Pub. No.: US 2025/0119291 A1
(43) Pub. Date: Apr. 10, 2025

(54) SYSTEMS AND METHODS FOR DATA TRANSFER

(71) Applicants: THE ROYAL INSTITUTION FOR THE ADVANCEMENT OF LEARNING / MCGILL UNIVERSITY, Montreal (CA); UNIVERSITÉ PARIS-PANTHÉON-ASSAS, Paris (FR)

(72) Inventors: Ghassan Mahmood Mohammed AL-SUMAIDAEE, Montréal (CA); Rami ALKHUDARY, Versailles (FR); Zeljko ZILIC, Verdun (CA)

(73) Assignees: THE ROYAL INSTITUTION FOR THE ADVANCEMENT OF LEARNING / MCGILL UNIVERSITY, Montreal (CA); UNIVERSITÉ PARIS-PANTHÉON-ASSAS, Paris (FR)

(21) Appl. No.: 18/910,982

(22) Filed: Oct. 9, 2024

Related U.S. Application Data
(60) Provisional application No. 63/589,194, filed on Oct. 10, 2023.

(51) Int. Cl. H04L 9/32; G16H 10/60; H04L 9/08

(52) U.S. Cl. CPC H04L 9/3213; G16H 10/60; H04L 9/0822

(57) ABSTRACT

Systems and methods that facilitate data access by multiple unconnected entities, each having access to a data storage system storing data relating to a specific topic. A computer-readable data file containing access details for the data is generated and stored elsewhere in the data storage system. A non-fungible token (NFT) is generated that is uniquely assigned to the topic of the data, and is recorded on a blockchain ledger. The NFT has a metadata file that is uniquely associated to that NFT and that contains a pointer to the computer-readable data file containing the access details. Thus, with access to the NFT, an entity can open the metadata file, follow the pointer to the computer-readable data file, use the computer-readable data file to obtain the access details, and access the data itself using those access details. In some embodiments, the access details and/or the data are encrypted.

### CROSS REFERENCE TO RELATED APPLICATIONS

This application claims priority from U.S. Provisional Patent Application No. 63/589,194, entitled “SYSTEMS AND METHODS FOR DATA TRANSFER”, filed Oct. 10, 2023, which is hereby incorporated by reference as if set forth in full in this application for all purposes.

### TECHNICAL FIELD

The present invention relates to data transfer between entities. More specifically, the present invention relates to systems and methods for secure, low-friction data access by multiple unconnected entities.

### BACKGROUND OF THE INVENTION

In many fields, vast quantities of data are now stored electronically. There is an inherent tension between preserving the integrity and security of that data and easily transferring that data to other parties/enabling access to that data by other parties.

As one non-limiting example, significant electronic healthcare records are maintained by healthcare providers (HCPs). However, given the sensitive subject matter, preserving the security of such records is critical. As such, different healthcare providers do not have access to each other's files. Further, the patient (i.e., the data subject) likewise has no access to the existing files. Thus, if a patient changes HCPs, or sees a specific HCP for a specific need, they must direct the new provider to request their files from the old provider, requiring administrative work and time, and potentially resulting in delays, errors, and/or data leaks (e.g., if the wrong patient's files are sent to the new provider). Further, common data transfer methods, such as email, are inherently insecure. As well, the fragmentation of data means that comprehensive, holistic care from multiple HCPs has a high administrative burden.

Numerous noteworthy strategies have been described in the literature to solve the prevailing problem of fragmentation of these systems (the so-called interoperability problem). Most of these strategies revolve around the use of centralized platforms, such as cloud infrastructure, to facilitate interconnectivity. However, these solutions have limited resilience to potential attacks, because they are vulnerable to a single point of failure. In areas where confidential and sensitive data is involved, especially in healthcare, such vulnerability is unacceptable. The emergence of Electronic Data Interchange (EDI) technologies has streamlined data sharing. Nonetheless, EDI technologies face challenges due to its limited validation capabilities, and the typical practice of sending EDI messages in batches can result in messages that contain incomplete or inconsistent data. Therefore, solutions are needed that seamlessly integrate healthcare systems while meeting the security requirements for the data they contain.

Blockchain technology has emerged as a solution to address the challenge of fragmentation inherent in various information systems. Functioning as a cohesive and secure database or data storage system, blockchain-based systems promote peer-to-peer interactions among multiple parties. The records stored within the blockchain framework are interconnected using robust cryptographic methodologies and arranged in a chronological sequence, thereby preventing retroactive alterations. These inherent attributes hold promise for enhancing data security within the healthcare domain, particularly in protecting patients' records.

Fragmentation within a single healthcare network can be effectively mitigated by implementing blockchain-based systems. However, an emerging challenge is that fragmentation is exacerbated when communication must extend beyond the boundaries of a single network. Currently, blockchain-oriented networks are reaching their limits in establishing direct interconnectivity, necessitating the use of intermediate platforms. These platforms predominantly either have centralized architectures or follow different and often incompatible protocols. This situation creates significant barriers to healthcare, especially for patients who want seamless integration into new healthcare networks.

As such, there is a need for systems and methods that promote interoperability of data systems without sacrificing data security or integrity, and that involve low administrative burdens and low friction.

### SUMMARY OF THE INVENTION

This document discloses systems and methods that facilitate data access by multiple unconnected entities, each having access to a data storage system storing data relating to a specific topic. A computer-readable data file containing access details for the data is generated and stored elsewhere in the data storage system. A non-fungible token (NFT) is generated that is uniquely assigned to the topic of the data and is recorded on a blockchain ledger. The NFT has a metadata file that is uniquely associated with that NFT and that contains a pointer to the computer-readable data file containing the access details. Thus, with access to the NFT, an entity can open the metadata file, follow the pointer to the computer-readable data file, use the computer-readable data file to obtain the access details, and access the data itself using those access details. In some embodiments, the access details and/or the data are encrypted. Steganographic methods may also be used to cryptographically embed the access details in the computer-readable data file.

In a first aspect, this document discloses system for data transfer between entities, said system comprising: a server for communicating with a data storage system, said data storage system being accessible to each of said entities, and said server comprising: a token-generation module configured to generate a non-fungible token (NFT) for recording in a blockchain ledger; and a metadata module configured to generate a metadata file uniquely corresponding to said NFT, said metadata file containing a pointer to a computer-readable data file stored in said data storage system, said computer-readable data file containing access details for data in said data storage system, said data only being accessible by way of said access details, and said metadata module being configured to store said metadata file in said data storage system, wherein said NFT immutably links to said metadata file, wherein said metadata file is amendable, wherein said access details are retrievable from said computer-readable data file, and wherein said data is added to said data storage system by one of said entities and a different one of said entities receives said NFT, such that said different one of said entities is able to retrieve said data by way of said metadata file, said computer-readable data file, and said access details, thereby enabling data transfer between said entities.

In another embodiment, this document discloses a system wherein said server further comprises an encryption module for encrypting said access details to thereby produce encrypted access details and wherein said computer-readable data file comprises said encrypted access details.

In another embodiment, this document discloses a system wherein said encryption module also generates a private key for decrypting said access details.

In another embodiment, this document discloses a system wherein said encryption module applies a steganography technique to embed said access details in said computer-readable data file.

In another embodiment, this document discloses a system wherein said computer-readable data file is an encrypted image file.

In another embodiment, this document discloses a system wherein said private key and said NFT are shared with a third party to enable said third party to retrieve said access details and to thereby access said data.

In another embodiment, this document discloses a system wherein said third party stores said access details in a third-party blockchain ledger, said third-party blockchain ledger being maintained by said third party.

In another embodiment, this document discloses a system wherein said data storage system is a distributed file storage system.

In another embodiment, this document discloses a system wherein, when said data in said data storage system is updated, said access details, said computer-readable data file, and said metadata file are amended.

In another embodiment, this document discloses a system wherein said data is medical data relating to a patient.

In a second aspect, this document discloses a system for data transfer between entities, said system comprising: a computer-readable data file containing access details for data in a data storage system, said data storage system being accessible to each of said entities and said data only being accessible by way of said access details; a non-fungible token (NFT) recorded in a blockchain ledger; a metadata file uniquely associated with said NFT, said metadata file stored in said data storage system and said metadata file containing a pointer to said computer-readable data file; and a server for communicating with said data storage system and said blockchain ledger and for generating and editing said metadata file, wherein said NFT immutably links to said metadata file, wherein said computer-readable data file is encrypted, wherein said access details are retrievable from said computer-readable data file, and wherein said data is added to said data storage system by one of said entities and a different one of said entities receives said NFT, such that said different one of said entities is able to retrieve said data by way of said metadata file, said computer-readable data file, and said access details, thereby enabling data transfer between said entities.

In a third aspect, this document discloses a method for transferring data between entities, said method comprising: receiving a non-fungible token (NFT); accessing a metadata file uniquely associated with said NFT, said metadata file containing a pointer to a computer-readable data file; accessing said computer-readable data file using said pointer, said computer-readable data file containing access details for data in a data storage system, said data only being accessible by way of said access details; and accessing said data by way of said access details, wherein said data is added to said data storage system by one of said entities and said method is practiced by a different one of said entities, thereby enabling data transfer between said entities.

In another embodiment, this document discloses a method wherein said access details contained in said computer-readable data file are encrypted and said method further comprises decrypting said access details after accessing said computer-readable data file.

In another embodiment, this document discloses a method wherein said computer-readable data file is an image file.

In another embodiment, this document discloses a method wherein said computer-readable data file is stored in said data storage system.

In another embodiment, this document discloses a method wherein said metadata file is stored in said data storage system.

In another embodiment, this document discloses a method wherein said data storage system is a distributed file storage system.

In another embodiment, this document discloses a method wherein said data is medical data relating to a patient.

### BRIEF DESCRIPTION OF THE DRAWINGS

The present invention will now be described by reference to the following figures, in which identical reference numerals refer to identical elements and in which:

FIG. 1 is a block diagram showing a system according to one aspect of the invention;

FIG. 2 is a schematic diagram showing an implementation of the system of FIG. 1;

FIG. 3 is another schematic showing an implementation of the system of FIG. 1;

FIG. 4 is a flowchart detailing a method according to one aspect of the present invention.

### DETAILED DESCRIPTION OF THE PREFERRED EMBODIMENTS

To better understand the present invention, the reader is directed to the listing of citations at the end of this description. For ease of reference, these citations and references have been referred to by their listing number throughout this document. The contents of the citations in the list at the end of this description are hereby incorporated by reference herein in their entirety.

The present invention provides systems and methods that facilitate data access by multiple unconnected entities with low administrative burden and high security. The entities have access to a shared data storage system, where data relating to a topic is stored. Accessing the data requires access details (e.g., a pointer, uniform resource locator (URL), filename or file path, password or key, and/or other locator or access tools). A computer-readable data file containing the access details is generated and stored in the data storage system, at a location separate from the location of the data itself.

A non-fungible token (NFT) is also generated that is uniquely assigned to the topic of the data. (As may be understood, the topic of the data may be a project, a human data subject such as a user or patient, a non-human data subject, etc.) The NFT is recorded on a blockchain ledger. The NFT has a metadata file that is uniquely associated with that NFT and that contains a pointer (i.e., location information) for the computer-readable data file containing the access details. The computer-readable data file may be any type of data file, such as an image file, text file, video or audio file, etc., as detailed further below. The NFT is recorded in a public blockchain ledger, such that anyone may access it.

Thus, with access to the NFT, an entity can open the metadata file, follow the pointer to the computer-readable data file, use the computer-readable data file to obtain the access details for the data itself, and access the data itself using those access details. This approach thus avoids the current need to coordinate between the data source/uploader and the data recipient(s). The data recipient(s) merely need access to the NFT to obtain access to the data. In embodiments where the NFT is recorded on a publicly accessible blockchain network, data access by the data recipient(s) thus involves very little administrative overhead, and no action by the data uploader/data source.

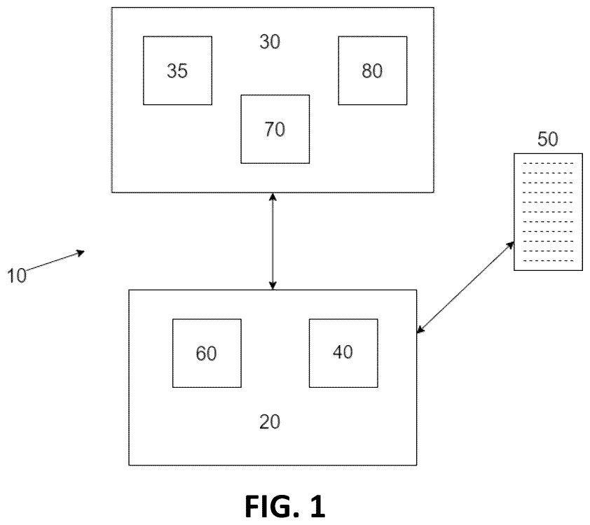

FIG. 1 is a block diagram showing a system 10 according to one aspect of the invention. A server 20 communicates with a data storage system 30 that contains data 35. The server 20 comprises a token-generation module 40 that generates a non-fungible token (NFT) for recording in a blockchain ledger 50. As described above, the NFT is uniquely assigned to the topic of the data 35. For example, in a healthcare context, each patient would have a unique NFT. The server 20 further comprises a metadata module 60 for generating and managing (e.g., amending) the metadata file 70 associated with the NFT. As well, after the metadata module 60 generates the metadata file 70, the server 20 stores the metadata file 70 in the data storage system 30 (at a location different from the location of the data 35). The metadata file 70 contains a pointer (i.e., location information) of a further file stored in the data storage system 30, namely the computer-readable data file 80. The access details in the computer-readable data file 80, as described above, can then be used to access the data 35.

The access details may have any suitable format, depending on the embodiment. As non-limiting examples, the access details may comprise text data, hypertext data, numerical data, and/or one or more files that may be encrypted and/or embedded within the computer-readable data file 80. The person skilled in the art would be able to select a format for the access details that would be suitable for any given implementation.

The metadata file 70 is, in some embodiments, a JSON file. Other file types may be used, depending on the embodiment. In particular, other file extensions such as YAML and XML may be used in some embodiments, as well as other types that be may suitable. JSON is currently the most convenient format for NFT metadata. As well, the content of the metadata file 70 may be human-readable, computer-readable, or a combination thereof. For example, the metadata file 70 may contain human-readable identifiers for the data subject/topic of the data 35, such as name, date of birth, etc. In some embodiments, further, the metadata file 70 itself is encrypted and/or password-protected, and/or contains encrypted and/or password-protected metadata.

The computer-readable data file 80 is created when the data uploader adds the data 35 to the data storage system 30 and may be manually or automatically created. For example, in some embodiments, a trained administrator of the data uploader generates the computer-readable data file 80 using the access details for the data 35 and manually stores the computer-readable data file 80 in the data storage system 30. In other embodiments, initially storing the data 35 in the data storage system 30 triggers an automatic generation and storage of the computer-readable data file 80 by the server 20 or by a different server or data processing unit separate from the system 20. Further, in some embodiments, the computer-readable data file 80 is generated by way of a smart contract operating on a blockchain network that has access to the data storage system 30. Again, as described above, the computer-readable data file 80 can be used to access the data 35.

It should be noted that, although FIG. 1 depicts the metadata file 70, the computer-readable data file 80, and the data 35 as being stored in the same data storage system 30, this is not required in all embodiments. In some embodiments, the metadata file(s) 70, computer-readable data file(s) 80, and data 35 may each be stored in different data storage systems. Alternatively, the data 35 may be stored in a separate system from the metadata file(s) 70 and computer-readable data file(s) 80, which may be stored together, or the metadata file(s) 70 and data 35 may be stored in a single system, or the computer-readable data file(s) 80 may be stored in a single system. As should be understood, the entities accessing/attempting to access the data will require access to each file and/or each data storage system used. However, there is no requirement that the various files and data be co-located or stored in a single data storage system 30. Many different file storage systems and mechanisms may be used in a single embodiment of the invention.

In a preferable embodiment, the access details of the data 35 change whenever changes are made to the data 35 (e.g., when the data 35 is altered, new data is added related to the same topic, or outdated or incorrect data is flagged or removed). Changing the access details ensures that the latest (most up-to-date) version of the data 35 will be retrieved by the data recipient(s). However, as should be understood, changes to the access details require commensurate changes to the computer-readable data file 80 and to the metadata file 70. That is, a new computer-readable data file 80 containing the new access details may be generated and stored in the data storage system 30 (depending on the embodiment), and, similarly depending on the embodiment, a new metadata file 70 pointing to the new computer-readable data file 80 may likewise be generated and stored. Otherwise, following the chain of pointers from the metadata file 70 to the computer-readable data file 80 to the data 35 would lead the data recipient to retrieve outdated data, or depending on the embodiment, to be unable to access the data and instead trigger an error.

This is particularly relevant when the access details are stored in a blockchain ledger by the data uploader. For example, as discussed above, private blockchain ledgers are becoming more commonly used in healthcare contexts. Such private ledgers (i.e., with access restricted to a single entity or cooperating network of entities) records the activities of patients with respect to that entity or cooperating network of entities. The private blockchain ledger can also serve as an immutable link between patients' activities and their medical data stored in a (private) storage system. One exemplary private ledger may be configured as follows: each data topic is assigned a “folder” F (i.e., a specific and unique data space) in the data storage system 30 used by the data uploader. (In a healthcare context, each folder may correspond to a single patient.) The folder F has a unique content identifier, F_{CID}. (“CID” meaning “content identifier”.) The folder contains records, each record having a unique identifier R_{CID}. Any changes in the records (e.g., the addition of new records, the removal or deletion of records, or the alteration of records) results in a change to the R_{CID}s within the folder. The F_{CID} is determined based on the R_{CID}s the folder contains, and thus any changes to the R_{CID}s also change the F_{CID}. In some embodiments, each F_{CID} and R_{CID} is recorded in a blockchain ledger as it is created and any changes to the F_{CID} and R_{CID} are likewise recorded in the blockchain ledger, such that there is an immutable record of each change. In some embodiments, the F_{CID}s are recorded in a blockchain ledger and the R_{CID}s are stored/maintained in other storage, including without limitation within the data storage system 30. The F_{CID} is, essentially, a file path/locator that can be used to access all of the records contained in that folder at a specific time. Thus, in this implementation, the F_{CID} comprises the access details and the records within the folder comprise the data 35. As should be understood, if the metadata file 70 and the computer-readable data file 80 are not changed when a new F_{CID} is generated, an incorrect/outdated F_{CID} could be erroneously used as the access details in an attempt to access the data. This could cause technical errors in the system or cause a user to extract and potentially rely on incorrect data.

Note, however, that, in some embodiments, updating the access details, the metadata file 70, and the computer-readable data file 80 may not be needed. For example, if the data 35 is simply overwritten in the data storage system 30, such that the access details will always allow access to the most-recent version of the data 35, the updating process described above may not be required.

Further, as should be understood, the updating process is preferably at least partially automated, to further reduce the administrative burden of data transfer. Thus, in some embodiments, the generation of new metadata files 70 and/or new computer-readable data files 80 occurs automatically. In other embodiments, new metadata files 70 and/or new computer-readable data files 80 require manual generation but once generated, a new metadata file 70 can be automatically associated with its NFT (i.e., the NFT's existing metadata file 70 is overwritten or amended to contain the new data).

As should be understood, the data storage system 30 is accessible to all entities (i.e., the entity uploading the data and the entity or entities seeking to access the data). In some embodiments, the data storage system 30 is publicly accessible—i.e., accessible to anyone with a suitably configured Internet connection. Such data storage systems may include peer-to-peer and/or distributed data storage systems, such as the InterPlanetary File System (IPFS) network. In other embodiments, the data storage system 30 is not entirely open/public but is shared between a specific network of entities. For example, the data storage system in some embodiments is a joint system operated and maintained by multiple healthcare networks, each of which also has their own, fully private, data stores. As would be understood, a data storage system that is fully private to a single entity would not be suitable for addressing the problem of data transfer between entities who do not already have shared data storage.

As well, the data storage system 30 is preferably able to store data of multiple data types, including without limitation: text data; image data; audio and/or video data; medical imaging or 4D data; computer-readable data such as machine code, that is not easily interpreted by humans; etc. As would be understood, the exact types of data collected depend on the nature of the topic.

The server 20, as should be understood, may comprise any suitable server architecture or hardware. For example, in some embodiments, the server 20 comprises a cloud-based or distributed server architecture, while in other embodiments the server 20 comprises a single server unit or a group of on-premise/in-house servers operated by a single entity. The most suitable server architecture for any implementation may be determined by the person skilled in the art.

As well, the blockchain ledger on which the NFT is recorded should also be accessible to all of the entities involved (i.e., the data source, the data recipient(s), and the data subject(s) as relevant), to allow each entity to access the NFT and its metadata file 70. As such, the ledger is preferably on a publicly accessible blockchain network, such as the well-known Ethereum network. However, in some embodiments, private or semi-private blockchain networks may be used.

Further, depending on the embodiment and the security/accessibility of the data storage system, additional security may be desired. For example, in healthcare settings, it is unacceptable to have access details for sensitive health information freely and publicly available to anyone with access to the NFT. Many other similar settings and contexts may be imagined in which the data should not be publicly accessible. As such, in some embodiments, the computer-readable data file may be an encrypted file. That is, the server in some embodiments comprises an encryption module that encrypts the access details according to a predetermined technique. When an authorized recipient follows the pointer in the metadata file to the computer-readable data file, then, that authorized recipient can decrypt the computer-readable data file and thereby obtain the access details. In a preferred embodiment, when the topic of the data is a human subject/patient, the encryption process generates a specific public/private key pair for decryption and the private key is provided to the human subject/patient. The public key of the key pair is used to encrypt the file or access data for the file and is associated with the NFT address for the human subject/patient such that a receiving network can verify, through a public smart contract, that the NFT presented to them belongs to the human subject/patient. The human subject/patient can then provide the private key to the intended data recipient(s), such as, e.g., a new healthcare provider, to enable their decryption of the data. In this way, the data subject can control who has access to their data.

In some embodiments, the computer-readable data file is a file in which the access details are embedded according to a steganographic method. Steganography is a well-known practice of hiding a message or information by concealing it within another message or within other information, such as within an image. Digital steganography can be used to hide information, such as data access details, within many forms of data, including text data, image data, audio data, video data, and network protocols for data transmission. Many other forms of data file can be used as the ‘carrier’ of the message, i.e., the computer-readable data file, as may be known in the art.

In some embodiments, the access details are both encrypted as ciphertext and then steganographically hidden. In such embodiments, the public key is associated with the NFT for the specific data subject, and used to encrypt the access details, as described above. The private key can then be used for decryption of the access details by the receiving network (after verifying the association between the NFT and the human subject, as described above). As well, in some embodiments, the data itself may also be encrypted and decryptable using the same or different keys as the keys used to encrypt and decrypt the access details. As would be understood, the complexity of the protections required depends on the nature of the data and of the requirements of the users. The person skilled in the art would understand the most suitable levels of encryption and encryption method(s) for a given implementation.

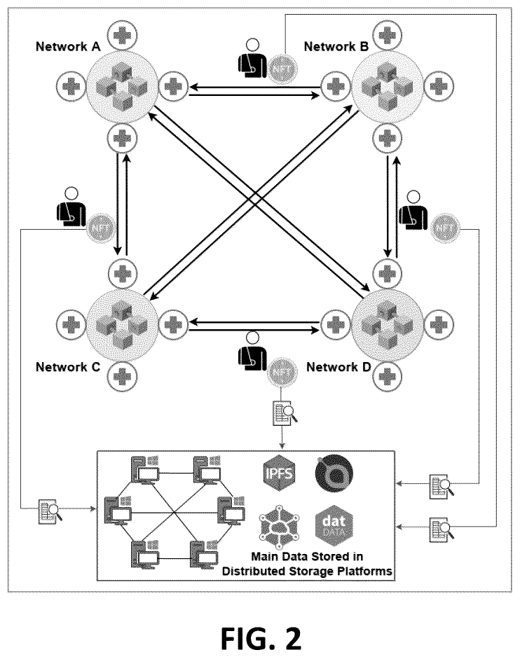

FIG. 2 is a schematic diagram showing a use of such a system, for a healthcare context. Multiple networks A, B, C, and D, which may not interact with each other under conventional arrangements, are each in contact with a data storage system such as IPFS (shown at the bottom of the diagram). When presented with an NFT by a patient (the icon of a male torso and head with a sling), each network is able to use the NFT to retrieve the patient's associated data from the data storage system. As should be understood, the data may be encrypted according to any suitable methods.

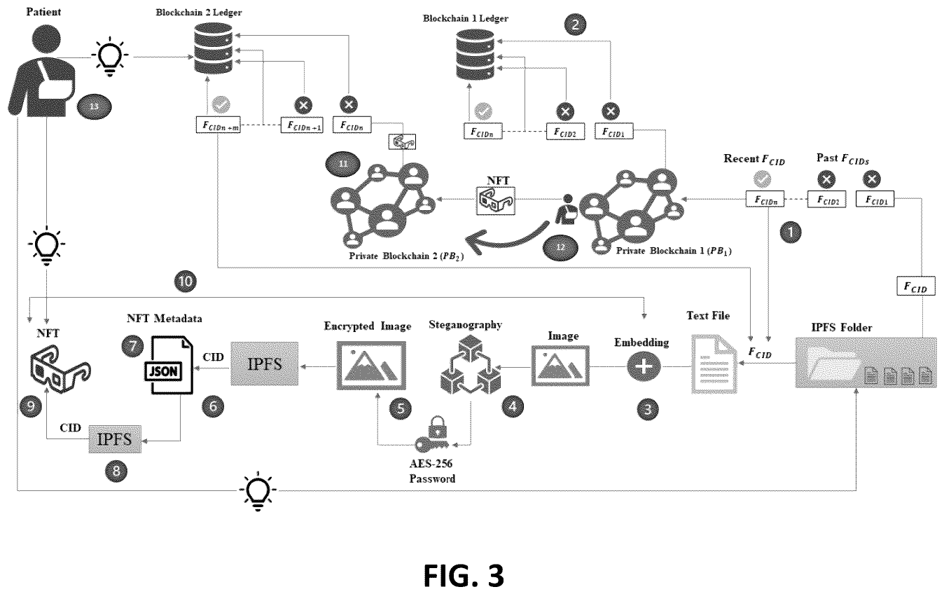

FIG. 3 is another schematic diagram showing another use of such a system. This implementation includes two separate healthcare entities, each of which uses a private blockchain ledger to track its own internal files (i.e., PB₁ and PB₂). Rather than being “transferred” between the entities, the data is stored in a folder in the data storage system (e.g., the IPFS network), and the folder is assigned a unique F_{CID} as described above, which is recorded in the data uploader's blockchain ledger PB₁. The F_{CID} (i.e., the access details of the data) is then transferred into a text file that is embedded within an image according to a steganographic method. (As described above, of course, the F_{CID} could be encrypted, or not, according to any suitable or desired method. The use of a steganographic image in this embodiment is merely one potential example, and should not be considered to limit the scope of this invention.) This encrypted image is stored in the data storage system and a pointer to the encrypted image is included in the metadata file (e.g., a JSON file) of an NFT assigned to the patient. The metadata file is then also stored in the data storage system. Thus (at top left of the diagram), if the patient wishes to provide their data to the second healthcare provider/entity, they merely provide the NFT (and any key or password necessary for decrypting the encrypted image to obtain the data access details). The second healthcare provider/entity can thus easily obtain the data access details and add them to its own blockchain ledger PB₂, without requiring direct access to the uploader's blockchain ledger PB₁ and without requiring intervention by any human administrator of the uploader's system. Further, the data is not exposed to leakage risk during data transfer over email or by other conventional means.

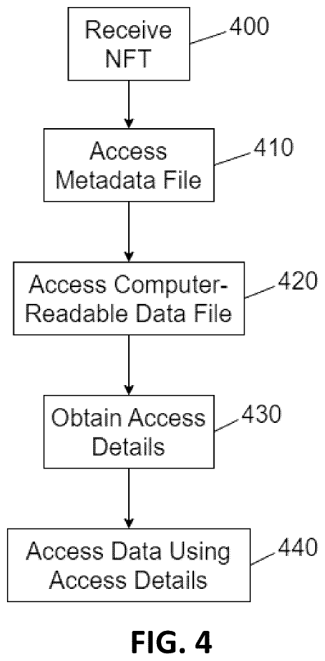

Referring now to FIG. 4, a flowchart detailing a method according to one aspect of the invention is shown. At step 400, an NFT is received by an entity that desires to access the data to which the NFT is assigned. At step 410, the metadata file uniquely associated with the NFT is accessed. The contents of the metadata file are used access the computer-readable data file at step 420. At step 430, the access details for the data are obtained from the computer-readable data file, as described above. The data is accessed using the access details, at step 440.

As noted above, for a better understanding of the present invention, the following references may be consulted. Each of these references is hereby incorporated by reference in its entirety:
[1] Ibrar Yaqoob et al. “Blockchain for healthcare data management: opportunities, challenges, and future recommendations”. In: Neural Computing and Applications (2021), pp. 1-16.
[2] Ghassan AI-Sumaidaee et al. “Performance analysis of a private blockchain network built on Hyperledger Fabric for healthcare”. In: Information Processing & Management 60.2 (2023), p. 103160.
[3] Ghassan AI-Sumaidaee et al. “Configuring Blockchain Architectures and Consensus Mechanisms: The Healthcare Supply Chain as a Use Case”. In: Blockchain Driven Supply Chains and Enterprise Information Systems. Springer, 2023, pp. 135-150.
[4] Ghassan AI-Sumaidaee et al. “A Technical Assessment of Blockchain in Healthcare with a Focus on Big Data”. In: 2022 IEEE International Conference on Big Data (Big Data). IEEE. 2022, pp. 2467-2472.
[5] Ghassan AI-Sumaidaee et al. “A Blueprint Towards an Integrated Healthcare Information System Through Blockchain Technology”. In HEALTHINFO 2021, The Sixth International Conference on Informatics and Assistive Technologies for Health-Care, Medical Support and Wellbeing. 2021.
[6] Ghassan AI-Sumaidaee et al. “Decentralized Storage for Big Data in Healthcare Between Reality and Ambition: IPFS and Sia”. in 2022 IEEE International Conference on Big Data (Big Data). Osaka, Japan: IEEE. December 2022, pp. 6578-6580. DOI: 10.1109/BigData55660.2022.10020670.

As used herein, the expression “at least one of [x] and [y]” means and should be construed as meaning “[x], [y], or both [x] and [y]”.

It should be clear that the various aspects of the present invention may be implemented as software modules in an overall software system. As such, the present invention may thus take the form of computer executable instructions that, when executed, implements various software modules with predefined functions.

Additionally, it should be clear that, unless otherwise specified, any references herein to ‘image’ or to ‘images’ refer to a digital image or to digital images, comprising pixels or picture cells. Likewise, any references to an ‘audio file’ or to ‘audio files’ refer to digital audio files, unless otherwise specified. ‘Video’, ‘video files’, ‘data objects’, ‘data files’ and all other such terms should be taken to mean digital files and/or data objects, unless otherwise specified.

Embodiments of the invention may be executed by a computer processor or similar device programmed in the manner of method steps or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g., “C” or “Go”) or an object-oriented language (e.g., “C++”, “java”, “PHP”, “PYTHON” or “C#”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for data transfer between entities, said system comprising:
a server for communicating with a data storage system, said data storage system being accessible to each of said entities, and said server comprising:
a token-generation module configured to generate a non-fungible token (NFT) for recording in a blockchain ledger; and
a metadata module configured to generate a metadata file uniquely associated with said NFT, said metadata file containing a pointer to a computer-readable data file, said computer-readable data file containing access details for data in said data storage system, said data only being accessible by way of said access details,
wherein said NFT immutably links to said metadata file,
wherein said metadata file is amendable,
wherein said access details are retrievable from said computer-readable data file, and wherein said data is added to said data storage system by one of said entities and a different one of said entities receives said NFT, such that said different one of said entities is able to retrieve said data by way of said metadata file, said computer-readable data file, and said access details, thereby enabling data transfer between said entities.

2. The system according to claim 1, wherein said server further comprises an encryption module for encrypting said access details to thereby produce encrypted access details and wherein said computer-readable data file comprises said encrypted access details.

3. The system according to claim 2, wherein said encryption module also generates a private key for decrypting said access details.

4. The system according to claim 2, wherein said encryption module applies a steganography technique to embed said access details in said computer-readable data file.

5. The system according to claim 2, wherein said computer-readable data file is an encrypted image file.

6. The system according to claim 3, wherein said private key and said NFT are shared with a third party to enable said third party to retrieve said access details and to thereby access said data.

7. The system according to claim 6, wherein said third party stores said access details in a third-party blockchain ledger, said third-party blockchain ledger being maintained by said third party.

8. The system according to claim 1, wherein said data storage system is a distributed file storage system.

9. The system according to claim 1, wherein, when said data in said data storage system is updated, said access details, said computer-readable data file, and said metadata file are amended.

10. The system according to claim 1, wherein said data is medical data relating to a patient.

11. A system for data transfer between entities, said system comprising:
a computer-readable data file containing access details for data in a data storage system, said data storage system being accessible to each of said entities and said data only being accessible by way of said access details;
a non-fungible token (NFT) recorded in a blockchain ledger;
a metadata file uniquely associated with said NFT, said metadata file containing a pointer to said computer-readable data file; and
a server for communicating with said data storage system and said blockchain ledger and for generating and editing said metadata file,
wherein said NFT immutably links to said metadata file,
wherein said computer-readable data file is encrypted,
wherein said access details are retrievable from said computer-readable data file, and wherein said data is added to said data storage system by one of said entities and a different one of said entities receives said NFT, such that said different one of said entities is able to retrieve said data by way of said metadata file, said computer-readable data file, and said access details, thereby enabling data transfer between said entities.

12. A method for transferring data between entities, said method comprising:
receiving a non-fungible token (NFT);
accessing a metadata file uniquely associated with said NFT, said metadata file containing a pointer to a computer-readable data file;
accessing said computer-readable data file using said pointer, said computer-readable data file containing access details for data in a data storage system, said data only being accessible by way of said access details; and
accessing said data by way of said access details,
wherein said data is added to said data storage system by one of said entities and said method is practiced by a different one of said entities, thereby enabling data transfer between said entities.

13. The method according to claim 12, wherein said access details contained in said computer-readable data file are encrypted and said method further comprises decrypting said access details after accessing said computer-readable data file.

14. The method according to claim 13, wherein said computer-readable data file is an image file.

15. The method according to claim 12, wherein said computer-readable data file is stored in said data storage system.

16. The method according to claim 12, wherein said metadata file is stored in said data storage system.

17. The method according to claim 12, wherein said data storage system is a distributed file storage system.

18. The method according to claim 12, wherein said data is medical data relating to a patient.
